(19) United States
(12) Patent Application Publication
(10) Pub. No.: US 2022/0017004 A1
(43) Pub. Date: Jan. 20, 2022

(54) LUMBAR SUPPORT DEVICE

(71) Applicant: Virtual Orthotics Pty Ltd, New South Wales (AU)

(72) Inventor: Phillip John HARTSHORNE, New South Wales (AU)

(21) Appl. No.: 17/352,913

(22) Filed: Jun. 21, 2021

(30) Foreign Application Priority Data
Jun. 22, 2020 (AU) 2020204165

(51) Int. Cl. B60N 2/66; A47C 7/46; G06F 30/10

(52) U.S. Cl. CPC B60N 2/66; G16H 10/60; G06F 30/10; A47C 7/46

(57) ABSTRACT

Lumbar support device 10 for a specific user seated on a specific seat, and a method for producing the lumbar support device 10. The lumbar support device 10 includes: a substantially rigid body 12 defining a peripheral region 14, a rear surface 16 having geometry defined by geometry of the specific seat, and an opposed front surface 18 having geometry defined by a lumbar region of the specific user; and a resiliently deformable layer 20 covering at least a portion of the front surface 18.

### CROSS-REFERENCE TO RELATED APPLICATIONS

This application claims priority to Australian Patent Application No. 2020204165, filed on Jun. 22, 2020, the disclosure of which are incorporated herein by reference in its entirety.

### TECHNICAL FIELD

The present disclosure relates, generally, to devices for supporting the lumbar region of a user whilst in a seated position and, particularly, to such a device configured to support the lumber region of a specific user seated on a specific seat.

### BACKGROUND

Back pain is a common musculoskeletal injury which causes discomfort and pain. Such injuries are often attributed to spending extended periods of time stationary in a seated position, for example, whilst working in an office, or driving a vehicle. Sitting for long periods causes fatigue in the muscles in the lumber region of the back, and surrounding abdominal muscles, allowing the spine to be displaced from its natural lordotic curvature. Such displacement can cause back, shoulder and/or neck pain, headaches, disc herniations and, over time, limit spinal mobility.

Lumbar support devices, such as cushions, rolls, and inflatable bladders, are often fitted to seats to attempt to alleviate this issue. Such devices are typically generic, intended to be used by a wide range of users. This means that the device often does not fill the space between the seat and the user's lumbar region consequently allowing some displacement of the spine which results in discomfort and pain.

Any discussion of documents, acts, materials, devices, articles or the like which has been included in the present specification is not to be taken as an admission that any or all of these matters were common general knowledge in the field relevant to the present disclosure as it existed before the priority date of each of the appended claims.

### SUMMARY

According to at least one disclosed embodiment, there is provided a lumbar support device for a specific user seated on a specific seat. The lumbar support device includes: a substantially rigid body defining a peripheral region, a rear surface having geometry defined by geometry of the specific seat, and an opposed front surface having geometry defined by a lumbar region of the specific user; and a resiliently deformable layer covering at least a portion of the front surface.

The lumbar support device may also include a cover arranged to cover the resiliently deformable layer and at least a portion of the body.

The rear surface of the body may be defined by a rim extending at least partially around the peripheral region. In such embodiments, the rim may discontinuously extend around the peripheral region to form a plurality of feet.

The resiliently deformable layer may be shaped such that its thickness tapers towards the peripheral region.

The body may define an operatively top end, bottom end and opposed sides, and wherein the taper of the resiliently deformable layer towards the bottom end defines a steeper gradient than a gradient of the taper towards the top end.

The taper of the resiliently deformable layer towards each of the sides may define an equal gradient.

The taper of the resiliently deformable layer towards each of the sides may define a gradient which is equal to or greater than the gradient of the taper towards the bottom end.

The body may be a unitary body which defines the rear surface and the front surface.

The lumbar support device may also include a belt secured to the body, the belt defining two ends and at least one end is associated with a fastening mechanism operable to releasably fasten the ends together to secure the device to the user.

According to at least one other disclosed embodiment, there is provided a method for producing a lumbar support device for a specific user seated on a specific seat, the method including: obtaining three-dimensional (3D) geometry data from geometry defined by the specific seat; obtaining 3D geometry data from geometry defined by a lumbar region of the specific user; fabricating a substantially rigid body to define a peripheral region, a rear surface having geometry defined by the 3D geometry data relating to the specific seat, and an opposed front surface having geometry defined by the 3D geometry data relating to the specific user; and mounting a resiliently deformable layer to the body to cover at least a portion of the front surface.

Obtaining the 3D geometry data relating to the specific user may include operating a 3D scanner to scan the lumbar region of the user. Scanning the lumbar region of the user may include scanning the lumbar region arranged in each of a plurality of positions relative to the specific seat, and wherein obtaining the 3D geometry data relating to the specific user includes averaging the 3D geometry data obtained from the plurality of scans.

Obtaining the 3D geometry data relating to the specific seat may include operating a 3D scanner to scan the seat.

The method may also include shaping the resiliently deformable layer such that its thickness tapers towards the peripheral region.

The body may define an operatively top end, bottom end and opposed sides, and shaping the resiliently deformable layer to taper towards the peripheral region may include shaping the taper towards the bottom end to define a steeper gradient than a gradient of the taper towards the top end.

Shaping the resiliently deformable layer to taper towards the peripheral region may include shaping the taper towards each side to define a gradient which is equal to or greater than the gradient of the taper towards the bottom end.

Throughout this specification the word “comprise”, or variations such as “comprises” or “comprising”, will be understood to imply the inclusion of a stated element, integer or step, or group of elements, integers or steps, but not the exclusion of any other element, integer or step, or group of elements, integers or steps.

It will be appreciated embodiments may comprise steps, features and/or integers disclosed herein or indicated in the specification of this application individually or collectively, and any and all combinations of two or more of said steps or features.

### BRIEF DESCRIPTION OF DRAWINGS

Embodiments will now be described by way of example only with reference to the accompany drawings in which:

FIGS. 1 and 2 are perspective, and side, exploded views, respectively, of a first embodiment of a lumbar support device;

FIG. 3 is a diagram of a user in three different seated positions;

FIGS. 4 and 5 are perspective and side views, respectively, of a three-dimensional (3D) computer model of part of the device shown in FIGS. 1 and 2;

FIG. 6 is a plan view of the rear of the lumber support device shown in FIGS. 1 and 2;

FIGS. 7 to 9 are perspective, end and side views, respectively, of a 3D computer model of another part of the device shown in FIGS. 1 and 2;

FIG. 10 is a plan view of the front of the lumber support device illustrated in FIGS. 1, 2 and 6; and

FIG. 11 is a plan view of a second embodiment of a lumber support device.

### DESCRIPTION OF EMBODIMENTS

In the drawings, reference numeral 10 generally designates a lumbar support device 10 for a specific user seated on a specific seat. The lumbar support device 10 includes: a substantially rigid body 12 defining a peripheral region 14, a rear surface 16 having geometry defined by geometry of the specific seat, and an opposed front surface 18 having geometry defined by a lumbar region of the specific user; and a resiliently deformable layer 20 covering at least a portion of the front surface 18.

The device 10 is intended to be used by a specific pilot whilst seated on a specific aircraft seat during flight. However it will be appreciated that the device 10 is suitable for use in other applications, such as by a driver of an automobile.

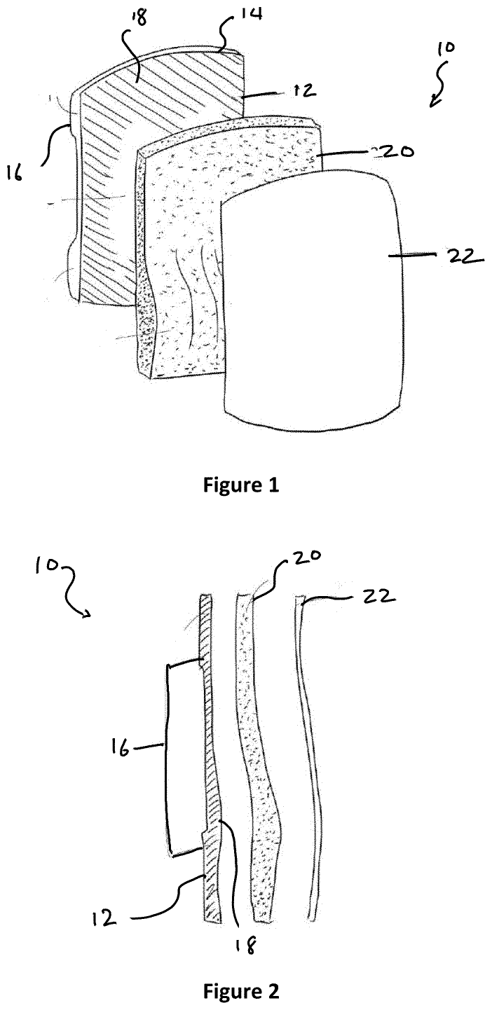

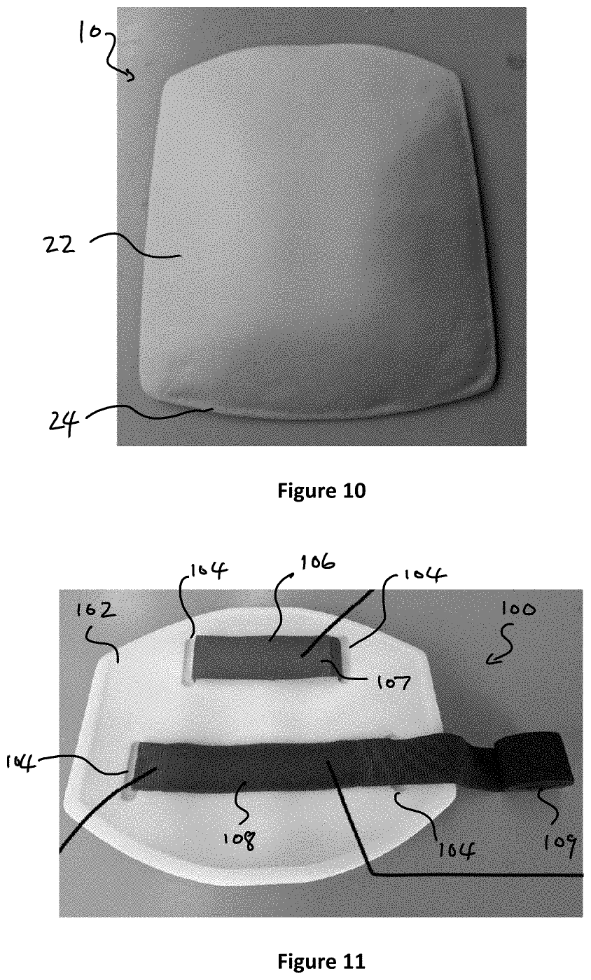

FIGS. 1 and 2 illustrate components of the device 10 including the body 12, the resiliently deformable layer 20 and, in this embodiment, a cover 22. The deformable layer 20 is mountable on the front surface 18 and joined to the body 12 by adhesive. The cover 22 is mountable on the deformable layer 20 and joined to the layer 20 by adhesive, and/or secured to the body 12 by stitching 24 (FIG. 10). In other embodiments (not illustrated), the cover 22 is absent.

In the illustrated embodiment the body 12 is configured as a unitary body, formed from a semi-rigid material, such as nylon, which allows some flex and defines the rear surface 16 and the front surface 18. The deformable layer 20 is configured as a foamed polymer sheet which covers the entire front surface 18. The cover 22 is a durable fabric, in this embodiment configured as a synthetic leather sheet which covers the deformable layer 20. In other embodiments (not illustrated) the body 12 is a multi-part construction comprising separate bodies which define the front surface 18 and the rear surface 16, respectively, and are connectable together to define the body 12. In further embodiments (not illustrated), the resiliently deformable layer 20 is formed as a compressible portion of the body 12, for example, by over moulding a resilient deformable material on to the body 12, or by configuring the structure of the layer 20 portion of the body 12 to allow compression.

Each of the rear surface 16 and the front surface 18 of the body 12 define a contour. As described in detail below, these contours are defined by the geometries of the specific user and the specific seat. The resiliently deformable layer 20 is a sheet of material having consistent thickness which is joined to the body 12 such that the layer 20 deforms to follow the contour of the front surface 18. The layer 20 is also shaped to enhance freedom of movement for the user, as described in detail below.

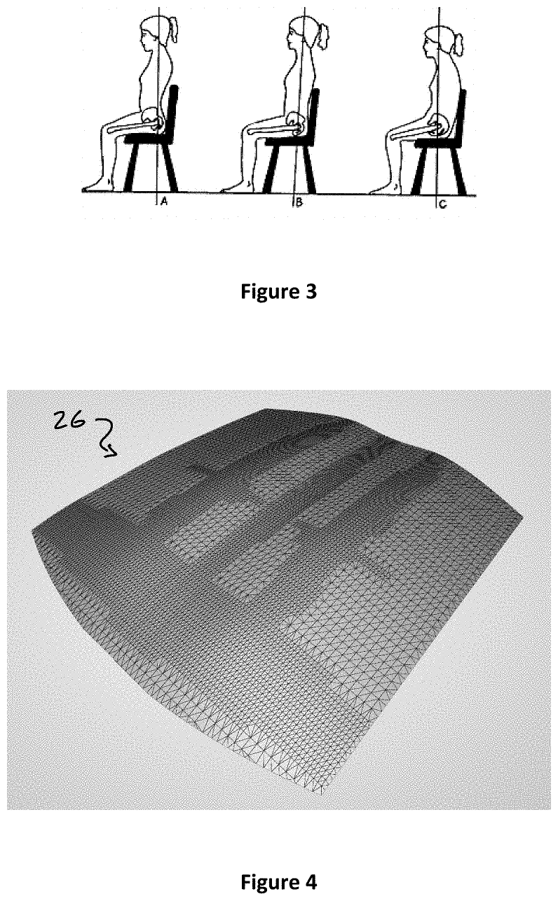

FIG. 3 is a diagram illustrating a user in three different seated positions. Position A illustrates a strained position where the shoulders are drawn backwards causing over-arching of the natural curvature of the spine. Position B illustrates a neutral, relaxed position with optimal spinal curvature. Position C illustrates a slouched position where the shoulders are hunched causing arching of the spine away from the natural curvature.

In some embodiments defining the geometry of the front surface 18 of the body 12 requires the specific user to assume each of the positions A-C relative to the specific seat, or a model representing the seat, to allow recording three-dimensional (3D) geometry data representing the user's lumbar region. Typically this involves operating a 3D scanner, such as a handheld structured light scanner, to scan the user's lumbar region whilst sitting in each of the positions A-C. Each scan records 3D geometry data corresponding with the geometry of the user's lumbar region.

The recorded 3D geometry data is processed by a computing device executing an algorithm. The algorithm is typically configured to average the 3D geometry data recorded at each of the positions A-C, and generate a 3D computer model 26 (FIG. 4) having geometry defined by the processed 3D geometry data. Averaging the 3D geometry data acquired in each of the positions A-C is useful as this means that the 3D model represents an average of the specific user's lumbar region geometry throughout the typical range of movement whilst seated in the specific seat.

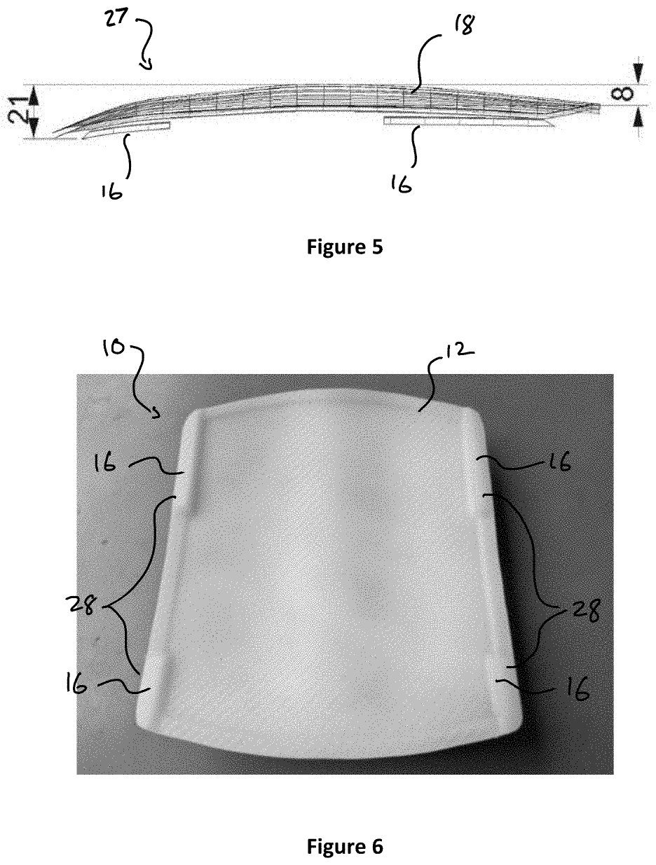

The geometry of the front surface 18 of the body 12 is defined by the geometry of the lumbar region model 26. Typically this involves the computing device generating a second 3D computer model 27 (FIG. 5) which has a portion, corresponding with the front surface 18, which defines inverse geometry to the geometry of the lumbar region model 26. Configuring the front surface 18 geometry in this way enhances fit of the device 10 to the user's lumbar region.

It will be appreciated that the front surface 18 geometry may be defined as a result of scanning the specific user's lumbar region arranged in more or less positions than described above. For example, in some embodiments the front surface 18 geometry is defined responsive to obtaining 3D data from the lumbar region arranged in a single position, such as position B. Also, in other embodiments (not illustrated) the geometry of the front surface 18 may be further refined by the computing device to incorporate other parameters, such as allowing a defined degree of flex by the body 12, and/or to smooth the front surface 18.

FIG. 5 shows a side view of the 3D computer model 27 which represents the body 12 illustrating the front surface 18 spaced from the rear surface 16 which, in this embodiment, is discontinuous. The rear surface 16 is shown as being arched to mirror the convex contour of the seat which this model 27 is configured to be used on.

FIG. 6 shows the rear of the device 10. In the illustrated embodiment the rear surface 16 of the body 12 is defined by a discontinuous rim which extends from the periphery of the rear of the body 12 to form four feet 28. Configuring the rear surface 16 in this way allows the body 12 to flex whilst in use, pressed against the specific seat. In other embodiments (not illustrated), the rear surface 16 is configured as a continuous rim around the periphery of the rear of the body 12. In yet other embodiments (not illustrated), the rear surface 16 is a continuous surface extending across the entire rear of the body 12.

In some embodiments the geometry of the rear surface 16 is defined due to recording 3D geometry data representing the specific seat. Typically this involves operating a 3D scanner to scan the seat. The recorded 3D geometry data is processed by the computing device to generate a 3D computer model (not illustrated) representing the seat. The computing device then defines geometry of the body model 27 to have a portion, corresponding with the rear surface 16, which is inverse to the seat model geometry.

In other embodiments the geometry of the rear surface 16 is defined due to obtaining 3D geometry data representing the specific seat from a database of seat geometries. For example, if the specific seat is a pilot's seat for a Boeing F/A-18E/F Super Hornet combat aircraft, model year 2007, the 3D geometry data relating to this seat may be selected from the database to allow the computing device to generate the rear surface 16 of the second 3D computer model 26. The database may be populated with the 3D geometry data obtained through 3D scanning of various seats and/or with 3D geometry data supplied by the manufacturer of the seat.

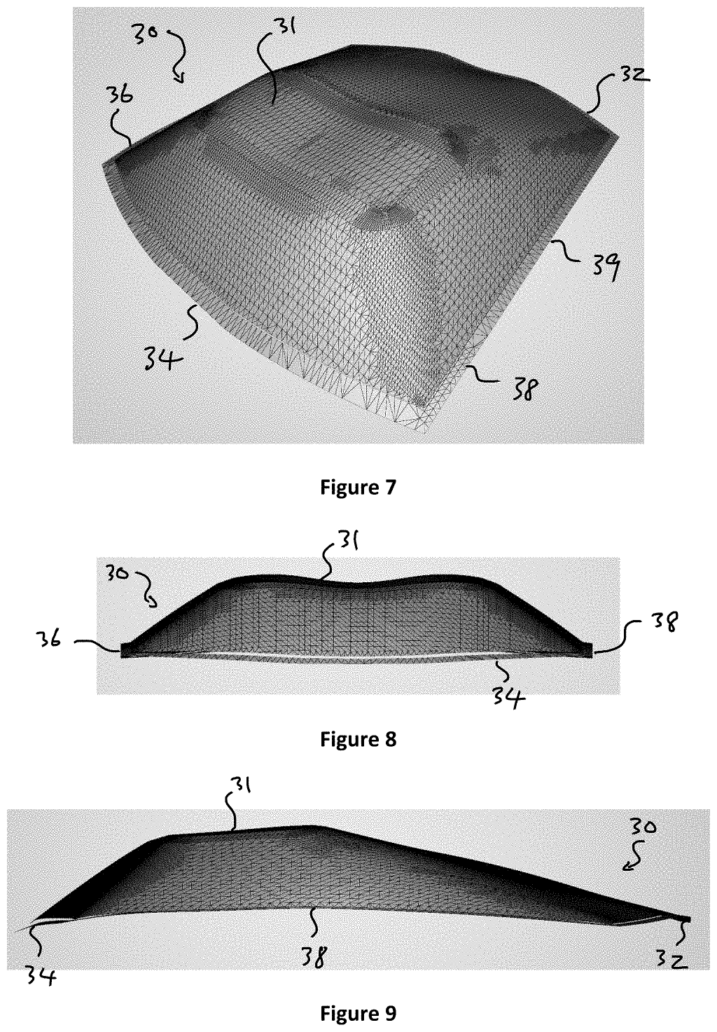

FIGS. 7 to 9 show another 3D computer model 30 representing the resiliently deformable layer 20. The model 30 defines a front surface 31 which is offset from the front surface 18 of the body 12 to define a complementary contour, and has an operatively top end 32, bottom end 34, and opposed sides, 36, 38. The front surface 31 is shaped to taper towards a peripheral region 39. Best shown in FIG. 9, the taper extending towards the top end 32 is more gradual than the taper extending towards the bottom end 34. The taper towards the bottom end 34 defines a steep gradient to limit or avoid displacing the user's sacrum. Best shown in FIG. 8, the taper extending towards each side 36, 38 is equal to each other. The taper towards each side 36, 38 defines a gradient equal to or greater than the taper towards the bottom end 34. The side-tapers are configured in this way to allow the user to comfortably turn towards each side 36, 38, for example, when looking over the user's shoulder whilst piloting an aircraft.

FIG. 10 shows the front of the device 10. In the illustrated embodiment the entire front surface 18 of the body 12, and the resiliently deformable layer 20, are covered by the cover 22. The cover 22 is configured as a synthetic leather sheet, such as polyurethane or polyvinyl chloride, which is secured to the body 12 by stitching 24 arranged around the periphery of the body 12.

FIG. 11 shows an alternative embodiment of the lumbar support device 100 which shares many features with the device 10, whereby common reference numerals indicate common features. The device 100 includes an alternatively configured body 102 which defines a plurality of slots 104. Two belts 106, 108, are arranged through the slots 104 and secured to form a loop. The first belt 106 has a hook and loop fastener arranged at one end 107 to allow fastening the end 107 to the belt 106. This belt 106 is arranged to be secured to the user's garments, for example, to an anti-gravity flight suit. The second belt 108 has a hook and loop fastener arranged at each end 109 to allow fastening the belt 108 around the user's waist.

Production of the device 10 involves: obtaining 3D geometry data from geometry defined by the specific seat; obtaining 3D geometry data from geometry defined by the lumbar region of the specific user; fabricating the body 12 to define a peripheral region, the rear surface 16 having geometry defined by the 3D geometry data relating to the specific seat, and an opposed front surface 18 having geometry defined by the 3D geometry data relating to the specific user; and mounting the resiliently deformable layer 20 to the body 12 to cover at least a portion of the front surface 18.

Obtaining the 3D geometry data relating to the specific seat typically involves operating a 3D scanner to scan the seat, as described above. Obtaining the 3D geometry data relating to the specific user typically involves operating a 3D scanner to scan the user's lumbar region, also as described above.

Fabricating the body 12 typically involves deriving computer instructions from the computer model 26 representing the body 12 and providing the instructions to a computer-controlled fabrication apparatus (not illustrated). This causes the apparatus to add or remove material in specific locations, defined by the instructions, to form an object having geometry which substantially corresponds with the geometry of the model 26.

In some embodiments, the apparatus is a multi-axis CNC milling machine. Providing the instructions to the milling machine causes milling (removal) of material from a block of the material. The material is removed from the specific locations until the fabricated object has the required body 12 geometry.

In other embodiments, the apparatus is a 3D printer, such as a fused filament fabrication (FFF) printer, or a sterolithography (SLA) printer. Providing the instructions to the 3D printer causes material to be added initially to a substrate, and then to previously fabricated material, in specific locations until the fabricated object has the required body 12 geometry.

In further embodiments a combination of the above approaches is employed. This may involve operating the 3D printer to produce a structure having geometry within a defined margin of the required body 12 geometry, and then operating the milling machine to refine the surfaces of the structure until the structure has the required body 12 geometry.

Fabricating the deformable layer 20 typically involves adhering a sheet of resiliently deformable material, such as a foam, to the front surface 18 of the body 12 such that the layer 20 conforms to the contour of the front surface 18, and then operating a tool to shape the layer 20 to form the tapers extending towards the periphery of the layer 20, as described above. Operating the tool may involve deriving computer instructions from the computer model 30 and providing the instructions to the milling machine to cause the milling machine to mill the layer 20, or manually operating a tool, such as a belt or disc sander.

Use of the device 10 involves trapping the device 10 between the specific user and the specific seat such that the rear surface 16 of the body 12 is pressed against the seat and the deformable layer 20 is pressed against the lumbar region of the user.

The device 10 is configured to have geometry which is complementary to geometry defined by the specific user, and geometry which is complementary to geometry defined by the specific seat. This means that the device 10 is shaped to form a close, secure fit between the lumbar region of the user and the seat on which the user is seated whilst using the device 10. The fit of the device 10 advantageously provides firm support to the user's lumbar region to mitigate displacement of the user's spine away from the natural lordotic curvature. This assists with reducing or preventing musculoskeletal injury.

It will be appreciated by persons skilled in the art that numerous variations and/or modifications may be made to the above-described embodiments, without departing from the broad general scope of the present disclosure. The present embodiments are, therefore, to be considered in all respects as illustrative and not restrictive.

1. A lumbar support device for a specific user seated on a specific seat, the lumbar support device including:
a substantially rigid body defining a peripheral region, a rear surface having geometry defined by geometry of the specific seat, and an opposed front surface having geometry defined by a lumbar region of the specific user; and
a resiliently deformable layer covering at least a portion of the front surface.

2. The lumbar support device of claim 1, further comprising a cover arranged to cover the resiliently deformable layer and at least a portion of the body.

3. The lumbar support device of claim 1, wherein the rear surface is defined by a rim extending at least partially around the peripheral region.

4. The lumbar support device of claim 3, wherein the rim discontinuously extends around the peripheral region to form a plurality of feet.

5. The lumbar support device of claim 1, wherein the resiliently deformable layer is shaped such that its thickness tapers towards the peripheral region.

6. The lumbar support device of claim 5, wherein the body defines an operatively top end, bottom end and opposed sides, and wherein the taper of the resiliently deformable layer towards the bottom end defines a steeper gradient than a gradient of the taper towards the top end.

7. The lumbar support device of claim 6, wherein the taper of the resiliently deformable layer towards each of the sides defines an equal gradient.

8. The lumbar support device of claim 7, wherein the taper of the resiliently deformable layer towards each of the sides defines a gradient which is equal to or greater than the gradient of the taper towards the bottom end.

9. The lumbar support device of claim 1, wherein the body is a unitary body which defines the rear surface and the front surface.

10. The lumbar support device of claim 1, also including a belt secured to the body, the belt defining two ends and at least one end is associated with a fastening mechanism operable to releasably fasten the ends together to secure the device to the user.

11. A method for producing a lumbar support device for a specific user seated on a specific seat, the method including:
obtaining three-dimensional (3D) geometry data from geometry defined by the specific seat;
obtaining 3D geometry data from geometry defined by a lumbar region of the specific user;
fabricating a substantially rigid body to define a peripheral region, a rear surface having geometry defined by the 3D geometry data relating to the specific seat, and an opposed front surface having geometry defined by the 3D geometry data relating to the specific user; and
mounting a resiliently deformable layer to the body to cover at least a portion of the front surface.

12. The method of claim 11, wherein obtaining the 3D geometry data relating to the specific user includes operating a 3D scanner to scan the lumbar region of the user.

13. The method of claim 12, wherein scanning the lumbar region of the user includes scanning the lumbar region arranged in each of a plurality of positions relative to the specific seat, and wherein obtaining the 3D geometry data relating to the specific user includes averaging the 3D geometry data obtained from the plurality of scans.

14. The method of claim 11, wherein obtaining the 3D geometry data relating to the specific seat includes operating a 3D scanner to scan the seat.

15. The method of claim 11, further comprising shaping the resiliently deformable layer such that its thickness tapers towards the peripheral region.

16. The method of claim 15, wherein the body defines an operatively top end, bottom end and opposed sides, and shaping the resiliently deformable layer to taper towards the peripheral region includes shaping the taper towards the bottom end to define a steeper gradient than a gradient of the taper towards the top end.

17. The method of claim 16, wherein shaping the resiliently deformable layer to taper towards the peripheral region includes shaping the taper towards each side to define a gradient which is equal to or greater than the gradient of the taper towards the bottom end.
